(19) United States
(12) Patent Application Publication
(10) Pub. No.: US 2016/0331779 A1
(43) Pub. Date: Nov. 17, 2016

(54) METHOD OF INACTIVATING TOXINS USING OXIDATIVE CHLORINE SPECIES

(71) Applicant: NovaBay Pharmaceuticals, Inc., Emeryville, CA (US)

(72) Inventors: John CREW, Emeryville, CA (US); Kathryn Najafi-Tagol, Emeryville, CA (US); Ron Najafi, Emeryville, CA (US); Dmitri Debabov, Emeryville, CA (US); Camron Noorbakhsh, Emeryville, CA (US); Timothy Shiau, Emeryville, CA (US); Lu Wang, Emeryville, CA (US)

(73) Assignee: NovaBay Pharmaceuticals, Inc., Emeryville, CA (US)

(21) Appl. No.: 15/151,344

(22) Filed: May 10, 2016

Related U.S. Application Data
(60) Provisional application No. 62/159,829, filed on May 11, 2015.

(51) Int. Cl. A61K 33/20; A61K 9/00

(52) U.S. Cl. CPC A61K 33/20; A61K 9/0048

(57) ABSTRACT

Toxins produced by bacteria promote infection and disease by directly damaging host tissues and by disabling the immune system. In one embodiment, the present application provides methods of treating and prevention further inflammation by inactivating toxins through use positive chlorinated ionic solution and or their derivatives.

### RELATED APPLICATION

This application claims the benefit of U.S. Provisional Application No. 62/159,829 filed May 11, 2015, which is incorporated herein in its entirety.

### BACKGROUND OF THE INVENTION

### Blepharitis

Blepharitis is characterized by inflammation of the anterior and posterior eyelid margins, but can also involve the surrounding skin, hairs, sebaceous glands, the mucocutaneous junction and the meibomian glands. It affects the conjunctiva, tear film and corneal surface, and causes dry eye indirectly by interfering with the secretion of lipids from the meibomian glands, which are holocrine glands that excrete the oily layer of the precorneal tear film. Blepharitis is a common ocular disorder and, to date, has no FDA-approved treatment for this condition.

Mild blepharitis usually remains undiagnosed and rarely managed. The prevalence of blepharitis was estimated at 10% in the general population by Claoue (Claoue, Eye, 1997, 11(6):865-868). Blepharitis is associated with a broad spectrum of ocular symptoms ranging from mild to persistent irritation, itching, burning, redness and ocular fatigue.

Acne rosacea, seborrheic dermatitis, psoriasis, atopy and hypersensitivity to bacterial products may all contribute to the etiology of blepharitis (Cher, Mod Med Austr., 1997, 52-62). It is generally assumed that infection plays a role in anterior blepharitis (Thygeson, 1946; Dougherty, 1984; Smith, CLAO J., 1995, 21(3):200-207), and cell-mediated immune responses to staphylococcal antigens has been emphasized (Ficker, Am J Opthalmol., 1991, 15; 111(4):473-479). However, studies have demonstrated that a small proportion of patients with meibomian gland dysfunction (Mathers, Cornea., 1996, 15(2): 110-119) and blepharitis have evidence of an active infection or show the production of staphylococcal toxins (Seal, Ophthalmology, 1990, 97(12): 1684-1688). Overall, while the pathophysiology of blepharitis is not well understood, current consensus is that bacteria, altered meibum lipid composition, and inflammation are all major contributors to the process.

Coagulase-negative staphylococci (C-NS) are regarded as normal flora of the lids and conjunctiva. The ability of these organisms to cause conjunctivitis and blepharitis can be overlooked or disregarded. Comparison of Staphylococcus sp. isolated from the conjunctiva and lids of 50 healthy volunteers with 248 strains of Staphylococcus isolated from patients with staphylococcal conjunctivitis or blepharitis demonstrated that S. epidermidis was the most frequent species isolated from the conjunctiva and lids of both groups. S. aureus was isolated only from infected patients. No individual C-NS species was found to be significantly associated with eye disease, but the colony count of C-NS after isolation was a useful indicator of conjunctivitis and blepharitis. The ability of Staphylococcus to ferment mannitol or mannose was associated with isolates only from infected patients (Au Y K, Jensen H G, Rowsey J, Reynolds M. Yan KeXueBao. 1993 September; 9(3):129-35).

### Necrotizing Fasciitis

Necrotizing fasciitis, commonly referred to in non-medical discourse as ‘flesh-eating’ infection, is a rapidly progressing infection of the deeper layers of the skin and subcutaneous tissues. Necrotizing fasciitis derives its name from the frequent spread of necrosis via fascial planes, and is a rare and life-threatening infection. Once diagnosed, necrotizing fasciitis is typically treated immediately with a combination of antibiotics, fasciotomy and extensive tissue debridement, and other supportive measures.

Many different aerobic and anaerobic bacteria can cause necrotizing fasciitis, including Streptococcus (Group A), Staphylococcus aureus (including methicillin-resistant strains), Vibrio vulnificus, Clostridium perfringens, Bacteroidesfragilis, Klebsiella, E. coli, and Aeromonashydrophila. Group A Streptococcus is considered the most common cause of necrotizing fasciitis (US CDC, 2014). Usually, infections from bacteria such as Group A Streptococcus are generally mild to moderate in severity and are easily treated; but sometimes toxins released by these bacteria can both destroy adjacent tissue and initiate an ineffectual and sustained immune system response which exacerbates tissue destruction (i.e., necrosis).

Exotoxins are proteins secreted by both Gram-positive and -negative bacteria. An exotoxin can cause damage to the host by destroying cells or disrupting normal cellular metabolism (Ray, 2010). All streptococcal and staphylococcal toxins are exotoxins.

Endotoxin is produced only by Gram-negative bacteria, and is lipid A of lypopolysacharide, an integral part of bacterial cell walls. Endotoxin may cause uncontrolled activation of mammalian immune systems with production of inflammatory mediators that may lead to septic shock (Kilar et al., 2013).

Some toxins released by bacteria can be categorized as ‘superantigens’ (SAgs), which can cause non-specific activation of T-cells and massive cytokine release (Reglinski and Sriskandan, 2014). SAgs are produced by pathogenic bacteria as a defense mechanism against the immune system. Compared to a normal antigen-induced T-cell response in which a very small fraction of T-cells are activated, SAgs are capable of activating up to 25% of T-cells. The large number of activated T-cells generates a massive immune response which is not specific to any particular epitope on the SAg, thus undermining one of the fundamental strengths of the adaptive immune system, i.e., its ability to target antigens with high specificity. More importantly, the large number of activated T-cells secrete large amounts of cytokines, the most important of which is interferon gamma (IFN-γ), which in turn activates macrophages. Consequently, activated macrophages over-produce pro-inflammatory cytokines such as IL-1, IL-6 and TNF-alpha. TNF-alpha is particularly important as a part of inflammatory responses, as in normal circumstances it is released locally in low levels and helps the immune system defeat pathogens. However when it is release systemically at high levels—in the case at hand, due to massive T-cell activation that results from Sags—it can cause severe and life-threatening symptoms, including shock and multiple organ failure.

Group A Streptococcus produces a number of superantigens, which are key virulence factors in the immunopathogenesis of invasive diseases caused by these bacteria.

These protein exotoxins have also been associated with severe group C and group G streptococcal infections. So many novel streptococcal superantigens have described recently that there is some confusion in their classification. A comprehensive classification of streptococcal antigens has been suggested by Commons et al. (2014).

Similarly, Staphylococcus aureus expresses several types of superantigens that corrupt the normal immune response and result in immunosuppression (Foster, 2005). Examples of staphylococcal superantigens include toxic shock syndrome toxin (TSST-1) and enterotoxins, of which there are six antigenic types (SE-A, B, C, D, E and G) (Llewelyn and Cohen, 2002). TSST-1 is a pyrogenic superantigen known to cause profound disturbances in the homeostasis of the immune system, including massive proliferation of T-cells and massive release of pro-inflammatory cytokines. TSST-1, as well as other superantigens, bind nonspecifically to major histocompatibility complex class II in the antigen-presenting cells and T-cell receptors bearing specific Vβ elements on the receptors. The structural stability of TSST-1 is critical in maintaining its function as a superantigen.

Toxins and superantigens released by Group A Streptococcus or other bacteria potentially underlying necrotizing fasciitis cause immune system dysfunction, promoting both local toxicity and necrosis and the absence of wound healing. Infections exacerbated by bacterial SAg release include septicemia, myositis, scarlet fever, necrotizing fasciitis, and Streptococcal toxic shock syndrome (STSS), as well as post-infectious sequelae, including acute rheumatic fever and post-streptococcal glomerulonephritis. Many studies have shown an association between SAgs and invasive disease or STSS.

In addition to the generation of toxins and superantigens, bacteria giving rise to necrotizing fasciitis release tissue-destructive enzymes, such as hyaluronidase and collagenase, which enable horizontal extension through deep fascial planes. Streptococcus and Staphylococcus pathogens use hyaluronidase as a virulence factor to destroy the polysaccharide that holds animal or human cells together, making it easier for the pathogen to spread through the tissues of the host organism (Starr and Engleberg, 2006). As this process progresses, thrombosis of the perforating nutrient vessels causes progressive dermis and skin ischemia, leading to bullae formation, ulceration, and skin necrosis (Maya et al., 2014).

Bacterial toxins, superantigens and enzymes released during the course of necrotic infections produce direct cytotoxic effects on surrounding tissues, while also causing immune system dysfunction, which is constituted by hyperactivation, suppression and dysregulation. As a result of the necrosis induced by all of these factors, necrotizing fasciitis often results in amputation of limbs or even death from toxic shock syndrome or sepsis if the toxins enter the bloodstream. Therefore, there is a need for an effective and safe method to concurrently treat both the direct bacterial cause of necrotizing fasciitis, as well as inactivate the bacterial toxins which may persist even after the bacteria have been killed.

### Bacterial Toxins

Many different aerobic and anaerobic bacteria can be isolated from eye lids and conjunctiva of blepharitis patients, including: Staphylococcus aureus, Staphylococcus epidermidis, Staphylococcus saprophyticus, other coagulase-negative staphylococci (C-NS), Corynebacterium spp. and Propionibacterium acnes. (Perry H D. The incidence and impact of blepharitis on cataract surgery. (Cataract & Refractive Surgery Toda, 2009). Other less frequently isolated anaerobes including other Propionibacterium spp., Peptococcus spp. and Peptostreptococcus spp. Veillonella sp., Fusobacterium spp., Bacteroides spp., Eubacterium spp., Lactobacillus spp., and Clostridium spp. were isolated very infrequently (Dougherty, 1984).

Exotoxins are proteins secreted by both Gram-positive and -negative bacteria. An exotoxin can cause damage to the host by destroying cells or disrupting normal cellular metabolism (Ray, 2010). All streptococcal and staphylococcal toxins are exotoxins.

Staphylococcus aureus expresses several types of superantigens that corrupt the normal immune response and result in immunosuppression (Foster, 2005). Examples of staphylococcal superantigens include toxic shock syndrome toxin (TSST-1) and enterotoxins, of which there are six antigenic types (SE-A, B, C, D, E and G) (Llewelyn & Cohen, 2002). TSST-1 is a pyrogenic superantigen known to cause profound disturbances in the homeostasis of the immune system, including massive proliferation of T-cells and massive release of pro-inflammatory cytokines. TSST-1, as well as other superantigens, bind nonspecifically to major histocompatibility complex class II in the antigen-presenting cells and T-cell receptors bearing specific Vβ elements on the receptors. The structural stability of TSST-1 is critical in maintaining its function as a superantigen.

Bacterial toxins, superantigens and enzymes released during the course of ocular infections produce direct cytotoxic effects on surrounding tissues, while also causing inflammation of the eye and surrounding area. Ocular bacterial infections are universally treated with antibiotics, which can eliminate the organism but cannot reverse the damage caused by bacterial products already present. For example, the three very common causes of bacterial keratitis—Pseudomonas aeruginosa, Staphylococcus aureus, and Streptococcus pneumoniae—all produce proteins that directly or indirectly cause damage to the cornea that can result in reduced vision despite antibiotic treatment. Most, but not all, of these proteins are secreted toxins and enzymes that mediate host cell death, degradation of stromal collagen, cleavage of host cell surface molecules, or induction of a damaging inflammatory response. Marquart & O'Callaghan J. Ophthalmol., 2013 Article ID 369094.

Toxins produced by the Staphylococcal spp. (in particular Staphylococcus epidermis and aureus) have been implicated as a major cause of blepharoconjunctivitis (McCulley J P. Blepharoconjunctivitis. Int Ophthalmol Clin. 1984; 24(2): 65-77), (Durand M L. Periocular infections. In: Mandell G L, Bennett J E, Dolin R, editors. Mandell, Douglas, and Bennett's Principles and Practice of Infectious Diseases. 7th ed. London: Elsevier, 2010:1569-1575), Groden L R, Murphy B. Rodnite J, Genvert G L Lid flora in blepharitis. Cornea. 1991:10(1):50-53). The Applicant has identified a need for an effective and safe method to treat blepharitis by killing relevant pathogens, as well as inactivate the bacterial toxins which may persist even after the bacteria have been killed.

### BRIEF DESCRIPTION OF THE BACKGROUND ART

Clark R A. J Immunol. 1986 June 15; 136(12):4617-22. Pneumolysin, a hemolytic toxin from Streptococcus pneumoniae, is a member of the group of thiol-activated, oxygen-labile cytolysins produced by various Gram-positive bacteria. The toxin activity of pneumolysin, as determined by lysis of 51Cr-labeled human erythrocytes, was destroyed on exposure to the neutrophil enzyme myeloperoxidase, hydrogen peroxide and a halide (chloride or iodide).

Valera M C, Maekawa L E, Chung A, Cardoso F G, Oliveira L D, Oliveira C L, Carvalho C A. Gen Dent. 2014 May-June; 62(3): 25-9. This in vitro study sought to evaluate the biomechanical preparation action on microorganisms and endotoxins by using sodium hypochlorite (NaOCl) and an intracanal medication containing Zingiberofficinale, with or without calcium hydroxide. Single-rooted teeth were contaminated, and root canal instrumentation (using 2.5% NaOCl) was performed. The results (submitted to Kruskal-Wallis and Dunn tests) showed that the NaOCl eliminated 100% of root canal microorganisms and reduced 88.8% of endotoxins immediately after biomechanical preparation, and 83.2% at 7 days after biomechanical preparation.

Maekawa L E, Valera M C, Oliveira L D, Carvalho C A, Koga-Ito C Y, Jorge A O. J Appl Oral Sci. 2011 April; 19(2):106-12. The purpose of this study was to evaluate the efficacy of auxiliary chemical substances and intracanal medications on Escherichia coli and its endotoxin in root canals. 2.5% NaOCl was able to eliminate E. coli from root canal lumen and reduced the amount of endotoxin compared to saline.

Martinho F C, Gomes B P. J Endod. 2008 March; 34(3):268-72. This clinical study was conducted to quantify endotoxins and cultivable bacteria in teeth with pulp necrosis and apical periodontitis before and after chemomechanical preparation with 2.5% sodium hypochlorite (NaOCl) and to investigate the possible correlation of endotoxin and cultivable bacteria with the presence of clinical symptomatology. Findings indicated that chemomechanical preparation with 2.5% NaOCl was moderately effective against bacteria but less effective against endotoxins in root canal infection. Furthermore, a statistically significant association was found between higher levels and clinical symptomatology.

Vincent F C, Tibi A R, Darbord J C. ASAIO Trans. 1989 July-September; 35(3):310-3. Bacterial bioproducts, i.e., endotoxins, are not removed by the highly porous membranes. Most studies have been done on the liquid phase of the dialysate. In this study, a biofilm has been made in a hemodialysis system, using an additional pump that continuously supplies several bioproducts: gram (+) or gram (−) bacteria, or extracted endotoxin. The activities of sodium hypochlorite, formaldehyde, and hydrogen peroxide are lower in the biofilm than in static studies.

U.S. Pat. No. 7,758,807 discloses methods and apparatus for achieving microbiological control, especially using active sources that generate hypochlorous acid vapor with reduced levels of chlorine vapor. These methods are effective in confined spaces and sealed containers. The active sources may be contained within permeable containers and may be actively dispersed. The active sources may be in the form of solids, liquids or gels.

U.S. Pat. No. 5,622,848 discloses amicrobiocidal solution for in vivo and in vitro treatment of microbial infections includes an electrolyzed saline containing regulated amounts of ozone and active chlorine species wherein the ozone content is between about 5 and 100 mg/L and the active chlorine species content of between about 5 and 300 ppm. The active chlorine species comprises free chlorine, hypochlorous acid and the hypochlorite ion as measured by a chlorine selective electrode. The solution may be used for the in vitro treatment of infected whole blood, blood cells or plasma to reduce contamination and is effective in treatment of fluids infected with HIV, hepatitis and other viral, bacterial and fungal agents.

U.S. Pat. No. 8,323,252 discloses a method of treating skin ulcers and related complications in patients by administering an oxidative reduction potential (ORP) water solution that is stable for at least twenty-four hours. The ORP water solution could also be treated as required for reducing the contents of pyrogens, endotoxins, or the like, that could be contaminating the solution.

United States Patent Publication No. 20110280854 discloses compositions and methods for treating or preventing E. coli infections. The compositions can be formulated as pharmaceutical compositions or as disinfectants, sanitizers, detergents or antiseptics, and can be used to eradicate or reduce E. coli populations and thereby treat or prevent infection by E. coli. A disinfectant or sanitizer as described herein can include one or more digestive enzymes, in various embodiments as described previously above, and can optionally include other active and inactive ingredients, including stabilizers (e.g., enzyme stabilizers), other disinfectants known to those having ordinary skill in the art, formulation excipients, colorants, perfumes, etc. One having ordinary skill in the art can select the additional active or inactive ingredients to include in a disinfectant. Examples of additional disinfectants include: sources of active chlorine (i.e., hypochlorites, chloramines, dichloroisocyanurate and trichloroisocyanurate, wet chlorine, chlorine dioxide etc.).

United States Patent Publication No. 20070264355 discloses binary methods and compositions comprising hypohalite (preferably hypochlorite) and peroxide (preferably hydrogen peroxide) directed to the killing of pathogenic microbes such as parasites, bacteria, fungi, yeast and prions, the oxidation of toxins, and the preparation of potable water. The binary methods and compositions extend the microbicidal potency of conventional hypochlorite by providing additional singlet molecular oxygen generated in situ, and offer more control over reactive chlorination exposure than hypochlorite alone. This combination is a highly effective disinfecting and decontaminating agent, capable of disinfection, detoxification, or deactivation of biological contamination and many chemical toxins, facilitating the sterilizing of surfaces and solutions, and the production of potable water.

Taylor H D, Austin J H. J Exp Med. 1918 March 1; 27(3):375-81. Dakin's hypochlorite and chloramine-T solutions will protect pigeons against multiple fatal doses of the toxin of Bacillus welch when the antiseptic and the toxin are mixed in vitro and allowed to stand in contact for 5 minutes before injection. 2. The detoxicating action of the solutions is demonstrable also in the presence of serum. 3. Phenol solution, 0.25 percent, has no such action.

Cole K D, Gaigalas A, Almeida J L. Biotechnol Prog. 2008 May-June; 24(3):784-91. The inactivation of the protein toxin ricin by the disinfectants bleach (sodium hypochlorite) and monochloramine was measured by the effect on mammalian cell cytotoxicity. Bleach caused a rapid inactivation of biological activity correlated with a rapid decrease in the fluorescence. Monochloramine required much higher concentrations for significant effects and the kinetics of the reactions were slow, with half-life values of the decrease on the order of minutes. Each protein showed individual differences in responses to the disinfectants, but there was a consistent correlation between the loss of fluorescence and the decrease in biological activity.

Yang C Y. Appl Microbiol. 1972 December; 24(6): 885-90. Cultures of Aspergillus flavus and aflatoxins were destroyed by a commercial bleach (Clorox; active ingredient, NaOCl) or analytical reagent grade NaOCl at 7.0×10⁻³ M NaOCl in 5 days. Addition of Clorox or NaOCl at 2.8×10⁻³ M to the fungal growth medium prior to inoculation completely inhibited the fungal growth. Aflatoxin production was inversely proportional to the logarithm of NaOCl concentration and time of treatment. Clorox and NaOCl were equally effective on aflatoxins, but fungal cells were lysed more readily by Clorox than by NaOCl. Mice and rats injected with aflatoxins and aflatoxins incompletely destroyed by Clorox died within 72 hr and had typical liver and kidney damage caused by aflatoxins. However, animals injected with NaOCl or Clorox or Clorox-destroyed aflatoxin extracts survived and showed no obvious liver or kidney damage.

### BRIEF SUMMARY OF THE INVENTION

In one embodiment, there is provided a method for the inactivation of a bacterial exotoxin in a patient, the method comprising an administration of a composition comprising an effective amount of hypochlorous acid or a hypochlorite salt to a tissue of the patient sufficient to inactivate the bacterial exotoxin. In one aspect of the above method, the tissue is the eye tissue or tissues surrounding the eye. In another aspect, the inactivation of the bacterial exotoxin is effective to treat blepharitis in the patient.

In one aspect of the above, the HOCl formulation is an aqueous solution at about 0.03%, about 0.04% and about 0.05%. In another aspect of the HOCl formulation, the pH is about pH 4, pH 4.5, pH 5, pH 5.5, pH 6, pH 6.5, pH 7, pH 8, pH 9 and pH 10.

In one aspect of the above, the method reduces the activity of the bacterial exotoxin in the patient. In another aspect, the method reduces the activity of the bacterial exotoxin on human cells or tissues.

In another aspect of the above method, the bacterial exotoxin is associated with an eye infection. In yet another aspect, the eye infection is associated with bacterial infection that is resistant to antibiotics. In another aspect of the above, the bacterial exotoxin is alpha-hemolysin toxin.

In another aspect of the above method, the bacterial exotoxin is selected from the group consisting of Staphylococcal enterotoxin, S. aureus alpha toxin, beta toxin, gamma toxin, Panton-Valentine leukocidin, TSST-1, enterotoxins A, B, C, D, E, H, J and K, exfoliative toxin A (ETA) and S. pyogenes streptokinase. In another aspect of the method, the hypochlorous acid is HOCl, and the hypochlorite salt is NaOCl. In another aspect, the bacterial exotoxin is Staphylococcal enterotoxin.

In one embodiment, the present application is directed toward compounds and compositions which show both antimicrobial activity as well as anti-toxin activity. The antimicrobial activity of hypochlorous acid and sodium hypochlorite has been well established. See Wang et al., 2007. However, these compounds additionally show unexpected inactivation of bacterial toxins. As used herein, inactivation means stopping the activity of the toxins or lipases.

Hypochlorous acid inactivates bacterial toxins (S. aureus alpha toxin, beta toxin, gamma toxin, Panton-Valentine leukocidin (PVL), TSST-1, enterotoxins A, B, C, D, E, H, J and K, exfoliative toxin A (ETA) and S. pyogenes streptokinase).

Other aspects of the current disclosure are described below.

Unless defined otherwise, all technical and scientific terms used herein have the same meaning as commonly understood by one of ordinary skill in the art. It is noted that as used herein and in the appended claims, the singular forms “a”, “and”, and “the” include plural referents unless the context clearly dictates otherwise. Thus, for example, reference to “the assay” includes reference to one or more assays and equivalents thereof known to those skilled in the art, and so forth.

“Blepharitis” refers to an inflammation that affects the eyelids. Blepharitis may involve a part of the eyelid where the eyelashes grow. Blepharitis may result in situations where the small oil glands located near the base of the eyelashes malfunction, which may lead to inflamed, irritated and itchy eyelids. It is known that some diseases and conditions can cause blepharitis. Blepharitis is known as a chronic condition that is difficult to treat.

“Effective amount” refers to the amount of an agent or composition as disclosed herein, such as HOCl that, when administered to a subject, surface or area for treating or preventing blepharitis or inactivating a toxin, is sufficient to effect such treatment or prevention, or inactivation. The “effective amount” will vary depending on the composition or formulation containing HOCl, the severity of the condition causing blepharitis, and the age, weight, etc., of the subject to be treated.

As used herein, “prevent”, “preventing” and “prevention” of treating blepharitis refer to reducing the risk of a subject from developing blepharitis, or reducing the frequency or severity of blepharitis in a subject.

“Treat”, “treating” and “treatment” of blepharitis refer to reducing the frequency or severity of symptoms of blepharitis (including eliminating them), or avoiding or reducing the chances of the occurrence of blepharitis.

### EXPERIMENTAL DESCRIPTION

S. aureus beta toxin, TSST-1, enterotoxins A, B, C, D, E, H, J and K may be obtained from Toxin Technology, Inc. (Sarasota, Fla.). S. aureus alpha toxin and S. pyogenesstreptokinase may be obtained from Sigma-Aldrich (St. Louis, Mo.). S. aureus gamma toxin and PVL may be obtained from IBT Bioservices (Gaithersburg, Md.).

Activity of Hypochlorous acid against alpha-hemolysin toxin of S. aureus was tested by cytotoxicity assay adapted from Crew, 2013, with some modifications. Human lung epithelial cells (A549, ATCC CCL 185) were grown in F12K cell culture medium supplemented with 10% fetal bovine serum (Life/Invitrogen, Carlsbad, Calif.) and 100 IU/mL penicillin/100 μg/mL streptomycin (MediatechInc, a Corning subsidiary, Manassas, Va.). Assays were performed using F12K medium with different concentrations of purified toxin by measuring the reduction of MTS tetrazolium compound into formazan that is soluble in cell culture medium.

Non-cytotoxic concentrations of HOCl and other products were determined by treating A549 cells with 1:10 serial dilutions and incubating treated cells for 16 hours at 37° C. with 5% CO₂. Dilutions that did not have an effect on cell viability were used with the subsequent assay involving alphahemolysin toxin inactivation. Cell viability was determined using a cell proliferation assay (CellTiter 96 Aqueous One Solution Cell Proliferation Assay [MTS], Promega, Madison, Wis.).

On the day before the assay, 5,000 cells/well were seeded in a 96-well plate, each well containing 100 μL of A549 cells in media. 1050±10 U/mL alpha-hemolysin toxin (Sigma Aldrich, St. Louis, Mo.) from S. aureus was incubated with a series of non-cytotoxic dilutions of hypochlorous acid for 1 hour. 50 μL from the wells of the drug plate were added to corresponding wells containing A549 cells in 100 μL media. The treated cells were incubated for 16 hours at 37° C. with 5% CO₂. At the end of the experiment, cell viability was determined with an assay (CellTiter 96 AQueous One Solution Cell Proliferation Assay [MTS], Promega, Madison, Wis.). 100 μL of PMS was added to 2.0 mL MTS and 20 μL was added to each well in the 96-well plate. The plate was incubated at 37° C. with 5% CO₂ for 4 hours. Cellular reduction of MTS into formazan was measured by obtaining absorbance at 490 nm with a SpectraMax M5 Microplate Reader. The half maximal effective concentration (EC₅₀) was calculated by plotting OD versus log of test article concentration in GraphPad Prism 4 using the sigmoidal dose-response equation. Inactivation of beta and gamma toxin can be determined using similar methods.

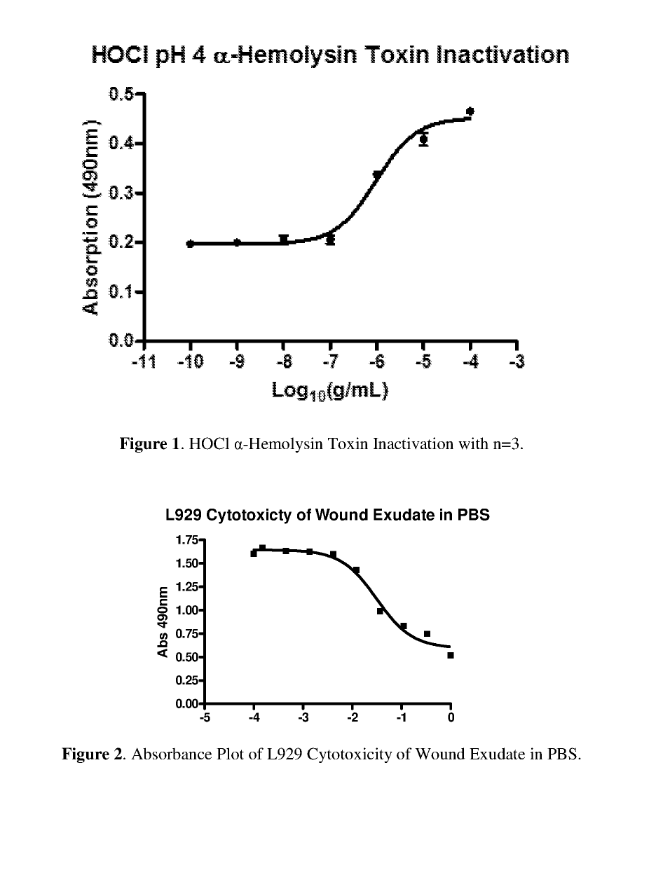

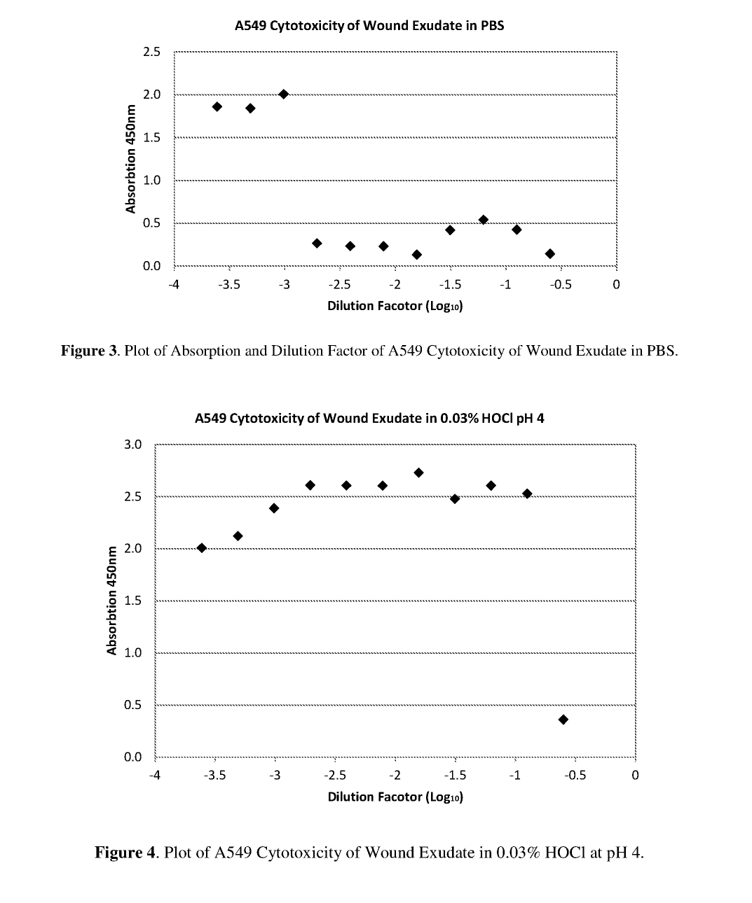

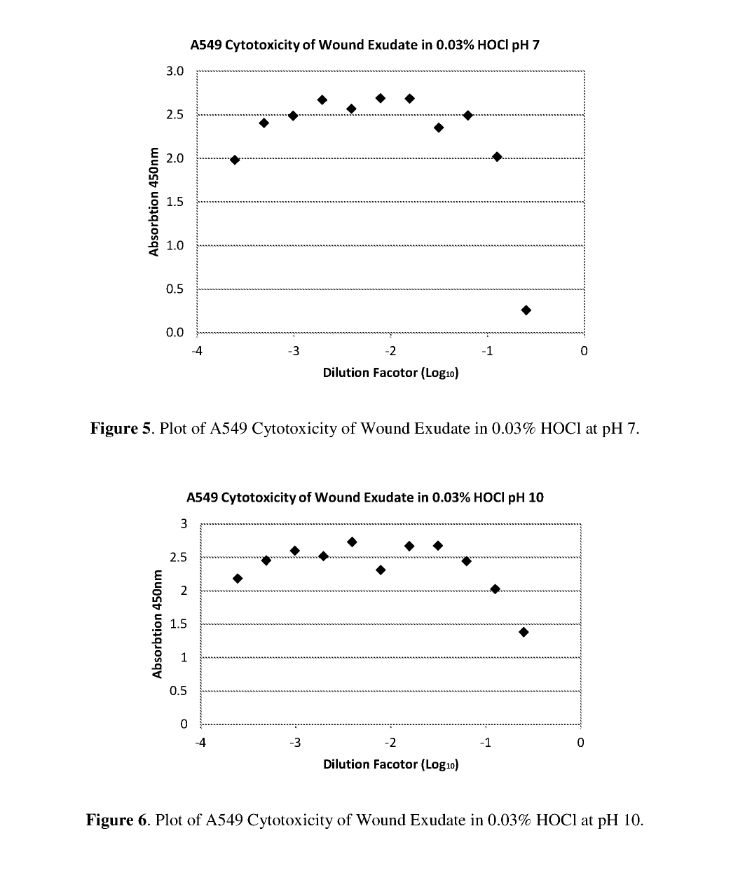

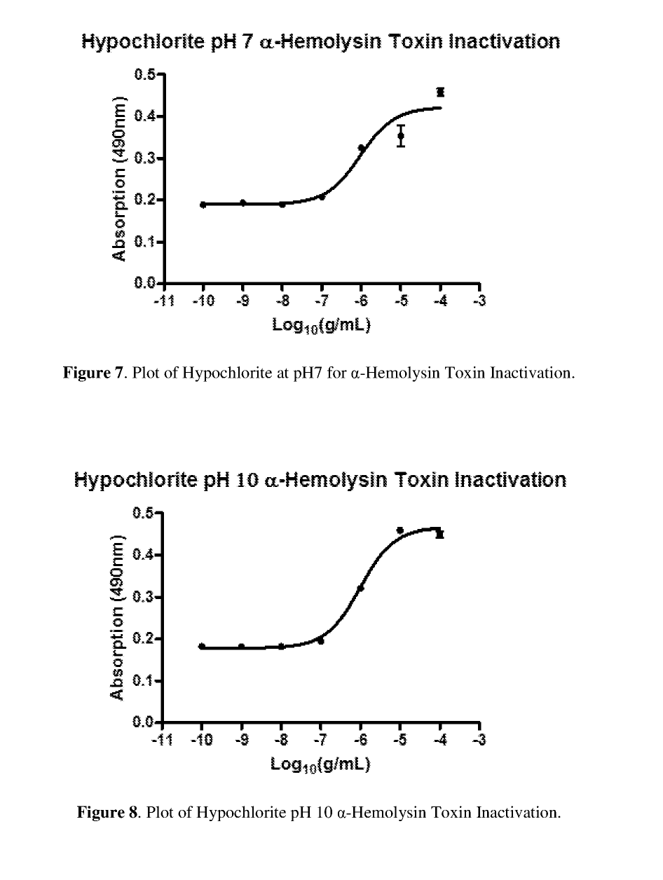

FIG. 1 shows one representative experiment of n=3. The EC₅₀ of HOCl pH 4 is 0.97±0.09 mg/mL.

It is believed that other toxins, especially toxins rich in disulfide bonds, will be inactivated in the presence of lower concentrations of hypochlorous acid, while other toxins not rich in disulfide bonds will not be inactivated at those concentrations.

Inactivation of streptokinase was determined by clot formation enzymatic assay (Sigma Aldrich, St. Louis, Mo.), whereby thrombin converts the soluble fibrinogen into soluble fibrin (Crew 2013). In the presence of streptokinase enzyme from S. pyogenes, the insoluble fibrin is converted into soluble fibrin fragments. Several hours before the study, streptokinase 175 U/mL) was incubated with the irrigation solution for 1 hour, followed by inactivation of excess irrigation solution by adding an equal volume of 20 mM methionine for 1 hour at room temperature.

In a separate glass tube, fibrinogen solution containing borate buffer, gelatin diluent, and plasminogen were mixed by swirling, and then equilibrated at 37° C. for 3 minutes. Irrigation solution and methionine treated streptokinase was added to the solution, mixed by swirling, and equilibrated at 37° C. for 1 minute prior to the addition of thrombin. The solution was mixed by swirling and equilibrated at 37° C. for approximately 2-3 minutes to allow for clot formation. A 4 mm glass bead was added to the top of the reaction mixture, and the time for the glassbead to touch the bottom of the tube was observed. In this clot assay, 0.001 μg/mL of the hypochlorous acid completely inactivated streptokinase, causing the fibrin to remain insoluble in solution, and the glass bead to remain at the top of the reaction mixture.

Hypochlorite inactivates bacterial toxins (S. aureus alpha toxin, beta toxin, gamma toxin, Panton-Valentine leukocidin, TSST-1, enterotoxins A, B, C, D, E, H, J and K, exfoliative toxin A (ETA) and S. pyogenes streptokinase).

Effect of hypochlorite (pH 7 and pH 10) on exotoxins was tested as described for HOCl pH 4. The EC₅₀ of hypochlorite pH 7 is 0.95±0.18 μg/mL. The EC₅₀ of hypochlorite pH 10 is 0.99±0.06 μg/mL.

Wound exudate from a NF patient is toxic to L929 cells.

Mammalian cell line L929 (mouse fibroblasts) was purchased from the ATCC. Wound exudates were collected from a collection container after NPWT irrigation of necrotizing fasciitis patients. The toxicity of wound exudates was evaluated after a 1-h exposure to serial dilutions of the exudates in PBS. Monolayers were grown to 80% confluence and treated with 2-fold dilutions of exudates in PBS (pH 7) for 1 h of incubation at 37° C. in a 5% CO₂ atmosphere. The exudate was then replaced with complete medium, and the plates were incubated overnight at 37° C. Cell viability was measured by using the Dojindo cell proliferation assay. The concentration of exudates cytotoxic to 50% of the cells (CT₅₀) was determined by using the GraphPad Prism program.

Wound exudate from a NF patient is toxic to A549 cells.

Mammalian cell line A549 (human lung epithelial) was purchased from the ATCC. Wound exudates was collected from a collection container after NPWT irrigation of necrotizing fasciitis patients. The wound exudate was exposed for 10 minute to an equal volume of PBS. The exudates and PBS mixture was then incubated at room temperature for 10 minutes in an equal volume of 20 mM L-methionine, for a total dilution of 1:4 of the original exudate. Monolayers were grown to 80% confluence and treated with 2-fold dilutions of exudate in PBS by 1 hour of incubation at 37° C. in a 5% CO₂ atmosphere. The exudate was then replaced with complete medium, and the plates were incubated overnight at 37° C. Cell viability was measured by using the Dojindo cell proliferation assay. Cytotoxicity to A549 cells was observed at a 1:512 dilution.

Wound exudate treated with hypochlorous acid is less toxic to A549 cells.

Wound exudate collected from a patient with necrotizing fasciitis was treated with 0.03% HOCl, at pH 4, 7 and 10. The wound exudate was exposed for 10 minute to an equal volume of HOCl. The HOCl was then neutralized by incubating for 10 minutes at room temperature in an equal volume of 20 mM L-methionine, for a total dilution of 1:4 of the original exudate. Cytotoxicity was measured the same way as described for wound exudate in PBS. Dilution of wound exudate cytotoxicity to A549 cells changed from 1:512 to 1:4 when exudates was pre-treated with 0.03% HOCl at pH 4, 7 or 10 indicating that HOCl neutralizes toxins in the exudate.

While the foregoing description describes specific embodiments, those with ordinary skill in the art will appreciate that various modifications and alternatives can be developed. Accordingly, the particular embodiments described above are meant to be illustrative only, and not to limit the scope of the invention, which is to be given the full breadth of the appended claims, and any and all equivalents thereof.

What is claimed:

1. A method for the inactivation of a bacterial exotoxin in a patient, the method comprising an administration of a composition comprising an effective amount of hypochlorous acid or a hypochlorite salt to a tissue of the patient sufficient to inactivate the bacterial exotoxin.

2. The method of claim 1, wherein the tissue is the eye tissue or tissues surrounding the eye.

3. The method of claim 1, wherein the inactivation of the bacterial exotoxin is effective to treat blepharitis in the patient.

4. The method of claim 3, wherein the bacterial exotoxin is associated with an eye infection.

5. The method of claim 4, wherein the eye infection is associated with bacterial infection that is resistant to antibiotics.

6. The method of claim 5, wherein the bacterial exotoxin is alpha-hemolysin toxin.

7. The method of claim 5, wherein the bacterial exotoxin is selected from the group consisting of Staphylococcal enterotoxin, S. aureus alpha toxin, beta toxin, gamma toxin, Panton-Valentine leukocidin, TSST-1, enterotoxins A, B, C, D, E, H, J and K, exfoliative toxin A (ETA) and S. pyogenes streptokinase.

8. The method of claim 7, wherein the hypochlorous acid is HOCl, and the hypochlorite salt is NaOCl.

9. The method of claim 7, wherein the bacterial exotoxin is Staphylococcal enterotoxin.
